(19) United States
(12) Patent Application Publication
(10) Pub. No.: US 2021/0333272 A1
(43) Pub. Date: Oct. 28, 2021

(54) BASE MATERIAL FOR PRODUCING SENSOR FOR ANALYSIS OF DETECTION TARGET, SENSOR FOR ANALYSIS OF DETECTION TARGET, AND METHOD FOR ANALYZING DETECTION TARGET

(71) Applicant: NATIONAL UNIVERSITY CORPORATION KOBE UNIVERSITY, Kobe-shi (JP)

(72) Inventors: Toshifumi TAKEUCHI, Kobe-shi (JP); Hirobumi SUNAYAMA, Kobe-shi (JP); Eri TAKANO, Kobe-shi (JP)

(21) Appl. No.: 17/242,073

(22) Filed: Apr. 27, 2021

(30) Foreign Application Priority Data
Apr. 28, 2020 (JP) 2020-079572

(51) Int. Cl. G01N 33/543; C08J 3/24

(52) U.S. Cl. CPC G01N 33/54386; C08J 2405/00; C08J 2333/26; C08J 3/245

(57) ABSTRACT

A measurement system capable of detecting a detection target such as sEVs with high specificity and rapidity, and also having both high stability and improved binding activity. A base material for producing a sensor for analysis of a detection target, including: a base material; and a polymer film provided on a surface of the base material, wherein the polymer film includes a concave that receives the detection target, and, inside the concave, a group for signal substance's binding and a polynucleotide group for nucleic acid aptamer's binding.

### FIELD OF INVENTION

The present invention relates to a technique for quickly detecting a detection target on a base material. More specifically, the present invention relates to a base material for producing a sensor for analysis of a detection target and a manufacture method of the same, a sensor for analysis of a detection target and a manufacture method of the same, and a method for analyzing a detection target.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is 34862232_1. TXT, the date of creation of the ASCII text file is Apr. 27, 2021, and the size of the ASCII text file is 2.92 KB.

### BACKGROUND

Small extracellular vesicles (sEVs) such as exosomes are one of endoplasmic reticula released from cells, and are lipid bilayer vesicles having a diameter of 20 to 200 nm. The sEVs contain a protein and nucleic acids such as miRNA and mRNA in their inside, and also have a protein on their surface. Since the sEVs are characterized by such substances, it is thought that, by analyzing the characteristics of the sEVs, it can be inferred what cells secreted the sEVs. In addition, the sEVs have been confirmed to exist in various body fluids, and can be collected relatively easily.

The sEVs secreted from cancer cells contain a tumor-derived substance. Therefore, it is expected that the diagnosis of cancer can be performed by analyzing substances contained in the sEVs in a body fluid. Furthermore, since sEVs are actively secreted by cells, they are expected to exhibit some characteristics even at the early stage of cancer.

Various methods for detecting exosomes have been reported. In particular, in the biosensor, which is described in WO 2018/221271 A, including a polymer film having a hole formed by a molecular imprinting technique, an antibody and a fluorescent molecules are selectively introduced into the hole in the polymer film. So, the biosensor enables specific antigen-antibody binding of the detection target exosomes in the hole and fluorescent detection thereof at the same time, and thus is useful as a sensor having excellent specificity and rapidity.

Antibodies greatly contribute to the performance of biosensors due to their excellent specificity and affinity. However, since antibodies are biomolecules, a biosensor using an antibody requires a certain amount of cost for production thereof, and needs to strictly control conditions such as temperature and pH during storage and use.

Here, nucleic acid aptamers are known as substances having a molecular recognition ability like antibodies. The nucleic acid aptamers can be chemically synthesized and thus inexpensively, and are highly stable against external stimuli.

On the other hand, as described in Nature Biotechnology volume 31, pages 453-457 (2013), there is a problem that a nucleic acid aptamer often has a lower affinity to a target molecule than an antibody. This problem is due to the fact that the antibody, which is a protein, comprises 20 kinds of amino acids as components, whereas the nucleic acid aptamer has only 4 kinds of bases as components. As a specific example, the dissociation constant Kd of an aptamer specific to human CD63 is only 17.1 nM, according to the data of “CD63 Aptamer Data Sheet”, [online], Apr. 1, 1998, BasePair Biotechnologies, Inc., [Searched: Mar. 25, 2020], Internet <URL: https://www.basepairbio.com/wp-content/uploads/2017/04/ATW0056-CD63-Aptamer-Data-Sheet_15Sept17.pdf>.

In order to improve the affinity of nucleic acid aptamers, studies have been conducted to increase the variation of bases by using a natural base into which a modifying group is introduced or an artificial base as a constituent base. Nature Biotechnology volume 31, pages 453-457 (2013) provides a technique for increasing the affinity of a DNA aptamer by incorporating an artificial base having properties different from those of a natural base, specifically, describes that the incorporation, as artificial base, of 7-(2-thienyl)imidazo[4,5-b] pyridine improved the affinity of a DNA aptamer by about 100 times.

### SUMMARY OF INVENTION

The use of a nucleic acid aptamer as a substance having a molecular recognition ability in a biosensor can be expected to reduce the production cost and improve the stability during storage and use, as compared with the case of using an antibody. However, considering that a nucleic acid aptamer composed of natural bases is often low in affinity, there was no option to use the nucleic acid aptamer composed of natural bases for the purpose of enhancing the binding activity of the biosensor.

At present, the technique for enhancing the binding activity of a nucleic acid aptamer having a low affinity depends exclusively on the introduction of an artificial base. When an artificial base is used as a component of a nucleic acid aptamer, normal cloning and sequencing, which are used in the production technique (SELEX method) for a nucleic acid aptamer consisting only of a natural base, cannot be performed, which involves the constraint of requiring a special method to identify the position of the artificial base from a random library. It is considered that the scope of biosensor techniques can be further expanded if the binding activity of the biosensor can be improved by other means that do not require such constraint.

Therefore, an object of the present invention is to provide a measurement system capable of detecting a detection target such as sEVs with high specificity and rapidity, and also having both high stability and improved binding activity.

When intentionally introducing a nucleic acid aptamer composed of natural bases in place of an antibody, in a biosensor including a polymer film having a hole formed by a molecular imprinting technique wherein an antibody and a fluorescent molecule are selectively introduced into the hole in the polymer film, the present inventor has found that the affinity thereof is increased to an unexpected level which is higher than that in the case of introducing the antibody. The present invention has been completed through further studies based on this finding.

The present invention encompasses a base material for producing a sensor for analysis of a detection target and a manufacture method of the same, a sensor for analysis of a detection target and a manufacture method of the same, and a method for analyzing a detection target. Specifically, the present invention provides the inventions of the following aspects.

Item 1. A base material for producing a sensor for analysis of a detection target, including:

a base material; and

a polymer film provided on a surface of the base material,

wherein the polymer film includes a concave that receives a detection target, and, inside the concave, a group for signal substance's binding and a polynucleotide group for nucleic acid aptamer's binding.

Item 2. The base material for producing a sensor for analysis of a detection target according to item 1, wherein the polynucleotide group for nucleic acid aptamer's binding has a length of 8 bases or more.
Item 3. The base material for producing a sensor for analysis of a detection target according to item 1 or 2, wherein the polynucleotide group for nucleic acid aptamer's binding is a single chain.
Item 4. The base material for producing a sensor for analysis of a detection target according to any one of items 1 to 3, wherein the polymer film is composed of a molecularly imprinted polymer prepared using the detection target or an object larger in size than the detection target as a template, and the concave corresponds to a part of a surface shape of the template.
Item 5. The base material for producing a sensor for analysis of a detection target according to any one of items 1 to 4, wherein the group for signal substance's binding is a thiol group.
Item 6. A sensor for analysis of a detection target including:

the base material for producing a sensor for analysis of a detection target according to any one of items 1 to 5;

a nucleic acid aptamer specific to the detection target, which is bound to the polynucleotide group for nucleic acid aptamer's binding; and

a signal substance which is bound to the group for signal substance's binding.

Item 7. The sensor for analysis of a detection target according to item 6, wherein the detection target is a microparticle having a membrane structure.
Item 8. The sensor for analysis of a detection target according to item 7, wherein the microparticle having a membrane structure is an extracellular vesicle.
Item 9. The sensor for analysis of a detection target according to any one of items 6 to 8, wherein the nucleic acid aptamer specific to the detection target has a specificity to a specific molecule expressed on a surface of the microparticle having a membrane structure.
Item 10. A method for analyzing a detection target, including:

a step of contacting a sample containing a detection target with the sensor for analysis of a detection target according to any one of items 6 to 9 to bind the detection target to the nucleic acid aptamer; and

a step of detecting a change in signal derived from the signal substance.

Item 11. A manufacture method of a base material for producing a sensor for analysis of a detection target, including:

a monomolecular film formation step (1-1) of forming on a base material a monomolecular film having a polynucleotide group for nucleic acid aptamer's binding and a polymerization initiating group on a surface thereof;

a template introduction step (1-2) of introducing a template having on a surface thereof a polynucleotide group capable of complementary binding with the polynucleotide group for nucleic acid aptamer's binding, into the polynucleotide group for nucleic acid aptamer's binding;

a surface modification step (1-3) of modifying a surface of the template with a polymerizable functional group via a reversible linked group;

a polymerization step (1-4) of forming a polymer film on a surface of the base material by adding a polymerizable monomer and synthesizing a molecularly imprinted polymer corresponding to a part of the surface of the template using the polymerizable monomer and the polymerizable functional group as substrates and the polymerization initiating group as a polymerization initiator; and

a removal step (1-5) of cleaving the complementary binding and the reversible linked group to convert respectively into a polynucleotide group for nucleic acid aptamer's binding and a group for signal substance's binding and removing the template.

Item 12. The manufacture method of a base material for producing a sensor for analysis of a detection target according to item 11, wherein the polynucleotide group for nucleic acid aptamer's binding has a length of 8 bases or more.
Item 13. The manufacture method of a base material for producing a sensor for analysis of a detection target according to item 11 or 12, wherein the polynucleotide group for nucleic acid aptamer's binding is a single chain.
Item 14. The manufacture method of a base material for producing a sensor for analysis of a detection target according to any one of items 11 to 13, wherein the template has, on a surface thereof, a reversible binding group capable of forming the reversible linked group by binding to the group for signal substance's binding, together with the polynucleotide group for nucleic acid aptamer's binding.
Item 15. The manufacture method of a base material for producing a sensor for analysis of a detection target according to any one of items 11 to 14, wherein the template is a silica particle.
Item 16. The manufacture method of a base material for producing a sensor for analysis of a detection target according to any one of items 11 to 15, wherein the group for signal substance's binding is a thiol group, and a reversible bond group capable of forming the reversible linked group by binding to the group for signal substance's binding is a thiol group.
Item 17. A manufacture method of a sensor for analysis of a detection target, including:

a step (1) of performing the manufacture method of a base material for producing a sensor for analysis of a detection target according to any one of items 11 to 16;

a step (2) of binding a nucleic acid aptamer specific to a detection target to the polynucleotide group for nucleic acid aptamer's binding by complementary binding; and

a step (3) of binding a signal substance to the group for signal substance's binding.

The present invention provides a measurement system capable of detecting a detection target such as sEVs with high specificity and rapidity, and also having both high stability and improved binding activity. That is, according to the present invention, a biosensor is constructed so that, in a polymer film having a hole formed by a molecular imprinting technique, a nucleic acid aptamer and a fluorescent molecule are selectively introduced into the hole, and thus a remarkably high affinity is achieved, as compared with that obtained in the case where an antibody is introduced into the hole, which makes it possible to detect the detection target with higher sensitivity.

The affinity achieved by the present invention could be more than 100 times higher than the affinity of the antibody, in some cases, even when a nucleic acid aptamer composed of natural bases was used as the nucleic acid aptamer. In view of the fact that the innate affinity of the nucleic acid aptamer composed of natural bases does not exceed the innate affinity of the antibody, but rather is often lower than the innate affinity of the antibody, it can be said that the affinity remarkably improved by the present invention, as compared with that in the case of introducing the antibody, is a surprisingly improved effect in view of the innate affinity of the nucleic acid aptamer. Although a specific mechanism by which such an effect is obtained is not clear, it is considered that, since nucleic acid aptamers are relatively small molecules, a plurality of nucleic acid aptamers can be arranged in a minute hole formed by a molecular imprinting technique, so that the detection target is captured by a plurality of bonds at the time of sensing, leading to a rise in the affinity of the nucleic acid aptamers per detection target to a surprising level. Furthermore, even if a high-affinity nucleic acid aptamer into which an artificial base is introduced is used as the nucleic acid aptamer, it can be expected that the innate affinity of the high-affinity nucleic acid aptamer is significantly improved.

### BRIEF DESCRIPTION OF DRAWINGS

FIG. 1 is a schematic diagram showing an example of a base material for producing a sensor for analysis of a detection target of the present invention;

FIG. 2 is a schematic diagram showing an example of a sensor for analysis of a detection target of the present invention;

FIG. 3 is a schematic diagram illustrating a monomolecular film formation step (1-1) in a manufacture method of a base material for producing a sensor for analysis of a detection target of the present invention;

FIG. 4 is a schematic diagram illustrating a template introduction step (1-2) following FIG. 3;

FIG. 5 is a schematic diagram illustrating a surface modification step (1-3) following FIG. 4;

FIG. 6 is a schematic diagram illustrating a polymerization step (1-4) following FIG. 5;

FIG. 7 is a schematic diagram illustrating a removal step (1-5) following FIG. 6;

FIG. 8 is a schematic diagram showing a manufacture method of a sensor for analysis of a detection target according to the present invention;

FIG. 9 is a schematic diagram illustrating an example of a method for analyzing a detection target of the present invention; and

FIG. 10 is a graph of the change in fluorescence intensity with respect to the concentration of sEVs, obtained in the detection of sEVs by fluorescence analysis using the sensor for analysis of the present invention in Example 3, and shows its curve fitting result.

### DETAILED DESCRIPTION OF THE PREFERRED EMBODIMENTS

[1. Base Material for Producing Sensor for Analysis of Detection Target]

The base material for producing a sensor for analysis of a detection target of the present invention is a base material that is a material for producing a sensor for analysis of the present invention which will be described below. This base material for producing a sensor for analysis is configured so that a user can easily customize the base material into a sensor capable of detecting a detection target such as a small extracellular vesicle with higher sensitivity.

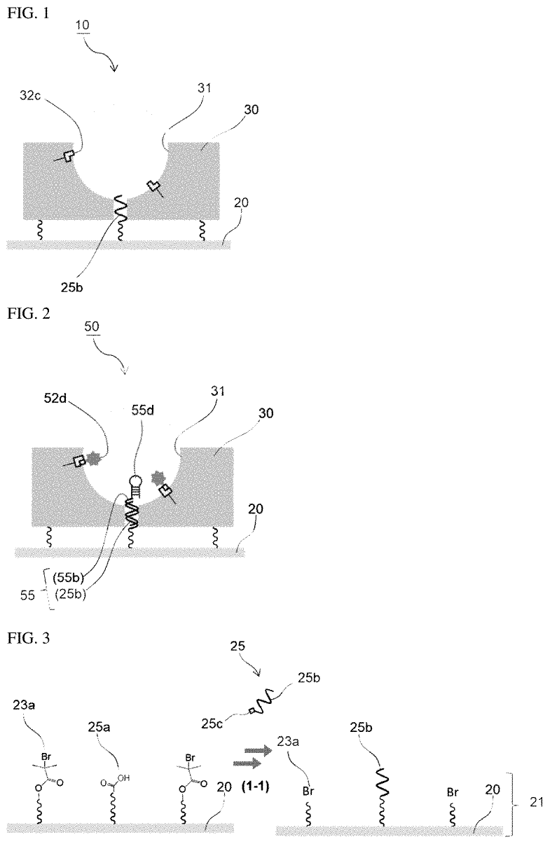

The base material for producing a sensor for analysis of a detection target according to the present invention includes a base material and a polymer film provided on a surface of the base material. The polymer film includes a concave that receives a detection target, and, inside the concave, a group for signal substance's binding and a polynucleotide group for nucleic acid aptamer's binding. FIG. 1 schematically shows an example of the base material for producing a sensor for analysis of a detection target according to the present invention. As shown in FIG. 1, a base material for producing a sensor for analysis 10 includes a base material 20 and a polymer film 30. The polymer film 30 is provided on a surface of the base material 20 and has a concave 31. The concave 31 is a hole formed in a size capable of receiving a detection target (a detection target 60 which will be described below). The base material for producing a sensor for analysis 10 has a polynucleotide group for nucleic acid aptamer's binding 25b and a group for signal substance's binding 32c in the concave 31. Hereinafter, the respective elements will be described in detail.

[1-1. Base Material]

The material for the base material 20 may be, for example, a material selected from the group consisting of metal, glass, and resin. Examples of the metal include gold, silver, copper, aluminum, tungsten, and molybdenum. Examples of the resin include poly (meth)acrylate, polystyrene, ABS (acrylonitrile-butadiene-styrene copolymer), polycarbonate, polyester, polyethylene, polypropylene, nylon, polyurethane, silicone resin, fluororesin, methylpentene resin, phenol resin, melamine resin, epoxy resin, and vinyl chloride resin.

The base material 20 may be formed by combining a plurality of materials selected from the above-indicated materials. For example, the base material 20 may be a base material in which a metal film is provided on a surface of glass or resin. The shape of the base material 20 may be a plate or particle shape. Preferred examples include gold base plates, glass base plates, gold nanoparticles, and silicon dioxide particles (silica particles, glass beads, etc.).

[1-2. Polymer Film]

The polymer film 30 is layered on the base material 20 and has a plurality of concaves 31. The concave 31 is a portion that serves as a sensor field in the sensor for analysis of a detection target of the present invention. The concave 31 is not limited as long as it is formed so as to be capable of receiving a detection target. For example, the concave 31 is preferably a molecularly imprinted polymer (MIP) formed using a molecularly imprinting method as will be described below. In this case, the concave 31 is formed by a template (template 40 which will be described below) used in the molecularly imprinting polymerization method, and has a shape corresponding to a part of the surface shape of the template. Since the concave 31 has only to be formed in a size capable of receiving a detection target, the template of the concave 31 may be a substance having the same size as the detection target, or a substance having a size larger than the detection target. Preferably, the template of the concave 31 is a substance having a size larger than that of the detection target.

“The concave 31 is formed in a size capable of receiving a detection target” means that, when a nucleic acid aptamer (nucleic acid aptamer 55d which will be described below) and a signal substance (signal substance 52d which will be described below) are bound to form a sensor for analysis (sensor for analysis 50 which will be described below), the size of the concave 31 opened on a surface of the base material 20 is sufficient to allow at least a part of a detection target to enter the concave 31 and approach the nucleic acid aptamer so that the detection target can be bound to the nucleic acid aptamer.

The opening diameter of the concave 31 is not particularly limited because it may vary depending on the detection target, and is, for example, 1 nm to 10 μm. Also, the thickness of the polymer film 30 is not particularly limited because it may vary depending on the detection target, and is, for example, 1 nm to 1 μm.

The polymer constituting the polymer film 30 may be, for example, a biocompatible polymer containing a biocompatible monomer-derived component. Biocompatibility refers to the property of not inducing the adhesion of biological materials. The polymer film 30 contains a biocompatible monomer-derived component, thereby making it possible to favorably suppress nonspecific adsorption in the polymer film 30. The biocompatible monomer is preferably a hydrophilic monomer, more preferably a zwitterionic monomer.

A zwitterionic monomer contains both an anionic group derived from an acidic functional group (for example, a phosphoric acid group, a sulfuric acid group, and a carboxyl group) and a cationic group derived front a basic functional group (for example, a primary amino group, a secondary amino group, a tertiary amino group, and a quaternary ammonium group) in one molecule. Examples of the zwitterionic monomer include phosphobetaine, sulfobetaine, and carboxybetaine.

More specifically, examples of the phosphobetaine include a molecule having a phosphorylcholine group in the side chain, and preferably include 2-methacryloyloxyethyl phosphorylcholine (MPC).

Examples of the sulfobetaine include N,N-dimethyl-N-(3-sulfopropyl)-3′-methacryloylaminopropaneaminium inner salt (SPB) and N,N-dimethyl-N-(4-sulfobutyl)-3′-methacryloylaminopropaneaminium inner salt (SBB).

Examples of the carboxybetaine include N,N-dimethyl-N-(1-carboxymethyl)-1-methacryloyloxyethaneaminium inner salt (CMB) and N,N-dimethyl-N-(2-carboxyethyl)-2′-methacryloyloxyethaneaminium inner salt (CEB).

Among these zwitterionic monomers, phosphobetaine is preferred, and 2-methacryloyloxyethylphosphorylcholine (MPC) is more preferred.

The proportion of the biocompatible monomer-derived component in the polymer film 30 is, for example, 10 mol % or more and 100 mol % or less. It is preferable that the content of the biocompatible monomer-derived component is the above lower limit or more, in order to suppress nonspecific adsorption on the surface of the polymer film 30. The lower limit of the range of the proportion of the biocompatible monomer-derived component is preferably 30 mol % or more, more preferably 50 mol % or more, still more preferably 70 mol % or more, even more preferably 80 mol % or more, even more preferably 90 mol % or more, particularly preferably 95 mol % or more.

[1-3. Polynucleotide Group for Nucleic Acid Aptamer's Binding]

The polynucleotide group for nucleic acid aptamer's binding 25b is a group that allows a nucleic acid aptamer (nucleic acid aptamer 55d which will be described below) to be introduced into the base material for producing a sensor for analysis 10 by complementarily binding the nucleic acid aptamer. As will be described in detail below, the nucleic acid aptamer 55d is introduced by complementary binding 55 between a polynucleotide group for introduction (polynucleotide group for introduction 55b which will be described below) extended to the nucleic acid aptamer 55d and the polynucleotide group for nucleic acid aptamer's binding 25b. The user can freely target a detection target, freely select the nucleic acid aptamer specific to the detection target to be targeted, and introduce it to the polynucleotide group for nucleic acid aptamer's binding 25b.

Further, the base material for producing a sensor for analysis 10 of the present invention can be customized so that the detection target can be analyzed using a plurality of kinds of nucleic acid aptamers in one base material 20, by introducing one kind of nucleic acid aptamer into one concave 31 and introducing other kinds of nucleic acid aptamers into other concaves 31 on one base material 20. A specific method of introducing a plurality of kinds of nucleic acid aptamers into one base material 20 is, for example, a method involving dividing a region on the polymer film 30 into a plurality of regions, and introducing different nucleic acid aptamers into the respective divided regions. Examples of the division method include a method of providing a portion where the polymer film 30 is absent, a method of providing a portion where the concave on the polymer film 30 is absent, and a method of providing a barrier so as to be projected on the polymer film 30. As a specific example of such customization, both a specific small extracellular vesicle and a specific protein can be analyzed on one base material 20 by introducing a nucleic acid aptamer that binds to the specific small extracellular vesicle into one concave 31, and introducing a nucleic acid aptamer that binds to the specific protein (protein not existing in the small extracellular vesicle membrane) into another concave 31. Further, as another specific example of customization, different kinds of surface proteins on a small extracellular vesicle can be analyzed on one base material 20, by introducing a nucleic acid aptamer that binds to a surface protein A of a specific small extracellular vesicle into one concave 31, and introducing a nucleic acid aptamer that binds to a surface protein B of the specific small extracellular vesicle into another concave 31. These specific examples can be applied not only to sEVs but also to other microparticles having membrane structures such as viruses, and can be applied not only to different kinds of surface proteins but also to different kinds of surface targets (for example, proteins and sugar chains).

In the schematic diagram given in the drawing, for the sake of convenience, one polynucleotide group for nucleic acid aptamer's binding 25b is present in one concave 31, but, actually, a plurality of polynucleotide groups for nucleic acid aptamer's binding 25b are present in one concave 31. On the surface of the base material 20, the polynucleotide group for nucleic acid aptamer's binding 25b does not exist in a portion other than the concave 31.

The polynucleotide group for nucleic acid aptamer's binding 25b is composed of a polynucleotide. The polynucleotide may be either DNA or RNA, but DNA is preferred from the viewpoint of stability. The base constituting the polynucleotide may be any of natural bases (adenine (A), guanine (G), cytosine (C), thymine (T) and uracil (U)) and artificial bases, but is, for example, a natural base from the viewpoint of easy manufacture and easy customization by the user.

The length of the polynucleotide group for nucleic acid aptamer's binding 25b is not particularly limited, but, from the viewpoint of obtaining a preferred complementary binding 55, is preferably 8 bases or more, more preferably 12 bases or more, still more preferably 16 bases or more, even more preferably 18 bases or more. Further, the upper limit of the range of the length of the polynucleotide group for nucleic acid aptamer's binding 25b is not particularly limited as long as the detection target can be captured in the concave 31. The upper limit is for example 25 bases or less, preferably 23 bases or less, more preferably 21 bases or less.

As the sequence of the polynucleotide group for nucleic acid aptamer's binding 25b, a sequence with which the recognition site of the nucleic acid aptamer to be introduced does not hybridize by mistake is appropriately selected. That is, the sequence of the polynucleotide group for nucleic acid aptamer's binding 25b has only to be designed so as not to resemble the nucleic acid aptamer, and may be composed of a random base sequence or a single nucleotide polymer as long as the sequence is designed so.

The polynucleotide group for nucleic acid aptamer's binding 25b is a single chain in the preferred aspect shown, but the polynucleotide group for nucleic acid aptamer's binding in the present invention is not limited to a single chain as long as the nucleic acid aptamer can be introduced by complementary binding, and is allowed to be a multiple chain such as a double chain or more.

[1-4. Group for Signal Substance's Binding]

The group for signal substance's binding 32c is a group that allows a signal substance (signal substance 52d which will be described below) to be introduced into the base material for producing a sensor for analysis 10 by binding the signal substance. The user can freely select the signal substance and introduce it into the group for signal substance's binding 32c.

Further, in the base material for producing a sensor for analysis 10 of the present invention, when different types of nucleic acid aptamers are introduced into one concave 31 and other concaves 31 in one base material 20, customization can be made so that different signal substances are introduced depending on the type of nucleic acid aptamer.

In the present invention, a plurality of groups for signal substance's binding 32c are usually provided per concave 31. The group for signal substance's binding 32c has only to be provided in a sufficient amount to detect the change in signal intensity at the time of sensing in the concave 31 of the sensor for analysis of the present invention (when the detection target is received in the concave 31). Therefore, the amount of the group for signal substance's binding 32 provided in one concave 31 is not particularly limited, and is, for example, about 1 to about 2000 per concave 31. However, the amount of the group for signal substance's binding 32 per one concave 31 is not limited to this, and can vary depending on the characteristics of the template, the polymer film thickness, the size of the concave 31 and/or the size of the target substance to be detected. On the surface of the base material 20, the group for signal substance's binding 32c is not substantially provided in a portion other than the concave 31.

The group for signal substance's binding 32c may be an irreversible binding group or a reversible binding group, and may be a covalent binding group or a non-covalent binding group. The reversible binding group is a group capable of constituting a reversible linked group by binding with any other reversible binding group (irrespective of whether covalent binding or non-covalent binding), and the term “reversible” means that the conversion (binding) from a reversible binding group to a reversible linked group and the conversion (cleavage) from a reversible linked group to a reversible binding group are bidirectionally possible.

The group for signal substance's binding 32c is preferably a reversible binding group, more preferably a covalent binding group. Examples of such a group include a thiol group (the corresponding reversible linked group is a disulfide group), an aminooxy group or a carbonyl group (the corresponding reversible linked group is an oxime group), a boronic acid group and a cis-diol group (the corresponding reversible linked group is a cyclic diester group), an amino group and a carbonyl group (the corresponding reversible linked group is a Schiff base), and an aldehyde group or ketone group and alcohol (the corresponding reversible linked group is an acetal group). A thiol group is preferred.

[1-5. Other Aspects]

The base material for producing a sensor for analysis of a detection target of the present invention has only to be configured so that at least one of the nucleic acid aptamer and the signal substance can be customized by the user. Thus, in another aspect, it is possible that a nucleic acid aptamer specific to the detection target has already been bound to the polynucleotide group for nucleic acid aptamer's binding 25b. In this case, the user can freely select and introduce the signal substance.

In still another aspect, it is possible that the signal substance has already been bound to the group for signal substance's binding. In this case, the user can freely target a detection target, freely select the nucleic acid aptamer specific to the detection target to be targeted, and introduce the nucleic acid aptamer.

[2. Sensor for Analysis of Detection Target]

The sensor for analysis of a detection target of the present invention includes: the base material for producing a sensor for analysis of a detection target described above; a nucleic acid aptamer specific to the detection target, which is bound to the polynucleotide group for nucleic acid aptamer's binding; and a signal substance which is bound to the group for signal substance's binding. FIG. 2 schematically shows an example of the sensor for analysis of a detection target of the present invention. As shown in FIG. 2, in the sensor for analysis 50, a nucleic acid aptamer 55d is bound to the polynucleotide group for nucleic acid aptamer's binding 25b, and a signal substance 52d is bound to the group for signal substance's binding 32c, inside the concave 31 provided in the polymer film 30 of the base material for producing a sensor for analysis 10.

[2-1. Detection Target]

The detection target (detection target 60 which will be described below) of the sensor for analysis of the present invention is not particularly limited in principle as long as it has a specificity to the nucleic acid aptamer 55d.

The chemical species as the detection target is not particularly limited, and examples thereof include low molecular weight organic compounds and high molecular weight compounds which are not derived from a living organism, and low molecular weight organic compounds and high molecular weight compounds which are derived from a living organism (including animals and plants). Among these chemical species, low molecular weight organic compounds and high molecular weight compounds which are derived from a living organism are preferred, and low molecular weight organic compounds and high molecular weight compounds which are derived from an animal are more preferred. Specific examples of such compounds include saccharides, lipids, proteins, peptides, nucleotides, and polynucleotides. Examples of the living organism include humans and non-human animals, and examples of the non-human animal include vertebrates, and mammals are preferred. Examples of the mammal include mice, rats, monkeys, dogs, cats, cows, horses, pigs, hamsters, rabbits, and goats.

Further, the functional species as the detection target is not particularly limited, and examples thereof include antigens, antibodies, receptors, disease markers, prions, and microparticles having a membrane structure. Further, when the detection target is a microparticle having a membrane structure, examples of the target molecule thereof include an antigen, an antibody, a receptor, and/or a disease marker expressed on the surface of the microparticle having a membrane structure; an antigen, an antibody, a receptor, and/or a disease marker expressed inside the membrane structure of the microparticle; and an antigen, an antibody, a receptor and/or a disease marker secreted by the microparticle having a membrane structure.

The disease marker is not particularly limited, and examples thereof include MUC-1, EpCAM, HER2, ERa, GGT1, CD24, PR, and many other tumor markers.

Examples of the microparticle having a membrane structure include extracellular vesicles, intracellular vesicles, organelles, viruses, and cells. Examples of the membrane structure include a lipid bilayer membrane structure. Examples of the extracellular vesicle include small extracellular vesicles (sEVs). The sEVs are particles released from cells and surrounded by a non-nucleus (non-replicable) lipid bilayer membrane, as defined by the International Society for Extracellular Vesicles (ISEV), and specific examples thereof include exosomes, microvesicles, and apoptotic bodies. Examples of the intracellular vesicle include lysosomes and endosomes. Examples of the organelle include mitochondria. Examples of the virus include influenza viruses (H1N1, H3N2, H5N1, H9N2, etc.), human immunodeficiency viruses, hepatitis viruses (HBV, HCV, etc.), measles viruses, rubella viruses, bovine viral diarrhea viruses, vaccinia viruses, Zika viruses, RS viruses, herpes viruses, Japanese encephalitis viruses, cytomegaloviruses, rabies viruses, human papilloma viruses, Ebola viruses, noroviruses (GII, GII.3, GII.4, etc.), rotaviruses, adenoviruses, dengue viruses, and coronaviruses (SARS coronavirus (SARS-CoV), SARS coronavirus-2 (SARS-CoV-2), etc.). Examples of the cell include cancer cells such as circulating tumor cells (CTC) and other disease-related cells. Among these microparticles having a membrane structure, extracellular vesicles and cells are preferred, and sEVs, viruses and cancer-related cells are more preferred.

Examples of the target expressed on the surface of a small extracellular vesicle (small extracellular vesicle-specific antigen or small extracellular vesicle antigen) include proteins such as CD63, CD9, CD81, CD37, CD53, CD82, CD13, CD11, CD86, ICAM-1, Rab5, Annexin V, LAMP1, EpCAM, and HER2; lipids (phospholipids such as phosphatidylserine and phosphatidylcholine); and sugar chains.

Examples of the target expressed on the surface or inside of a virus include spike (S) glycoprotein, envelope (E) protein, membrane (M) protein, hemagglutinin esterase (HE) protein, nucleocapsid (NC) protein, and non-structural (NS) protein.

Examples of the target expressed on the surface of a cancer cell (cancer cell-specific antigen) include proteins such as Caveolin-1, EpCAM, FasL, TRAIL, Galectine3, CD151, Tetraspanin 8, EGFR, HER2, RPN2, CD44, and TGF-β; lipids (phospholipids such as phosphatidylserine and phosphatidylcholine); and sugar chains.

As will be described below, when the signal substance 52d is configured as one of the fluorescent dye pair that causes fluorescence resonance energy transfer (FRET), the detection target 60 is bound to the other of the fluorescent dye pair in advance.

[2-2. Nucleic Acid Aptamer (Nucleic Acid Aptamer Specific to Detection Target)]

As the nucleic acid aptamer 55d, a nucleic acid aptamer having a specificity to the detection target is selected. The nucleic acid aptamer is a nucleic acid molecule having a relatively short (e.g., 20 to 200 bases length) base sequence, which has a specificity to a predetermined target. The binding mode of specific binding between the detection target and the nucleic acid aptamer 55d is not limited, and examples thereof include chemical bonds such a covalent bond, an ionic bond, a hydrogen bond, and electric adsorption; and a physical bond such as shape-dependent engagement.

Examples of the nucleic acid aptamer 55d used in the present invention include RNA aptamers, DNA aptamers, and DNA-RNA hybrid aptamers (DNA/RNA chimera aptamers). From the viewpoint of stability, the nucleic acid aptamer 55d used in the present invention is preferably a DNA aptamer. The base constituting the nucleic acid aptamer 55d may be any of natural bases (adenine (A), guanine (G), cytosine (C), thymine (T), and uracil (U)) and artificial bases.

In the present invention, since the affinity of the nucleic acid aptamer per detection target can be remarkably improved, even if the nucleic acid aptamer has an innate affinity that is equal to or lower than the affinity of the antibody or lower than the affinity of the antibody, it is possible to effectively obtain the effect of improving the affinity of the nucleic acid aptamer per detection target. From such a viewpoint, examples of suitable constituent bases of the nucleic acid aptamer 55d in the present invention include natural bases. It is preferable that the constituent bases of the nucleic acid aptamer 55d are natural bases from the viewpoints of easy acquisition, easy manufacture, and easy customization by the user. The innate affinity of the nucleic acid aptamer means an affinity when the nucleic acid aptamer is fixed on a flat surface or is in a non-fixed free state, not in the concave 31 like the sensor for analysis 50 of the present invention. The affinity is a value measured using a binding constant or a dissociation constant.

Of course, in the present invention, for the purpose of obtaining a further improved affinity, a nucleic acid aptamer containing an artificial base of which the innate affinity has been improved may be used as the nucleic acid aptamer 55d.

The base sequence of the nucleic acid aptamer 55d and the steric structure of the molecule are determined by those skilled in the art according to the detection target. As the nucleic acid aptamer 55d, a known nucleic acid aptamer can be used, and a new nucleic acid aptamer obtained by any known method can also be used. The method for obtaining the nucleic acid aptamer is not particularly limited, and any known method can be used. Typical examples of such a method include SELEX method (a method involving contacting a target with an oligonucleotide library containing a large number of oligonucleotides having a random sequence, selecting a group of oligonucleotides having a high affinity for the target, and amplifying the selected oligonucleotides to confirm whether or not they specifically bind to the target molecule).

Specific examples of the nucleic acid aptamer 55d include:

CACCCCACCTCGCTCCCGTGACACTAATGCTA (SEQ ID NO: 1) as a CD63-specific nucleic acid aptamer;

GCAGTTGATCCTTTGGATACCCTGG (SEQ ID NO: 2) as a MUC-1 specific nucleic acid aptamer;

CACTACAGAGGTTGCGTCTGTCCCACGTTGTCATGGGGGGTTGGCCT G (SEQ ID NO: 3) as an EpCAM-specific nucleic acid aptamer;

GGGCCGTCGAACACGAGCATGGTGCGTGGACCTAGGATGACCTGAG TACTGTCC (SEQ ID NO: 4) as an HER2-specific nucleic acid aptamer; and

ATACCAGCTTATTCAATTCGTTGCATTTAGGTGCATTACGGGGGTTATC CGCTCTCTCAGATAGTATGTGCAATCA (SEQ ID NO: 5) as an ERa-specific nucleic acid aptamer.

Other examples of the specific nucleic acid aptamer 55d include DNA or RNA aptamers reported, for example, in the review article on the application of aptamers to virus detection and antiviral therapy (X. Zoul, J. Wul, J. Gul, L. Shen, L. Mao, Application of Aptamers in Virus Detection and Antiviral Therapy, Front. Microbiol. 2019, 10, 1462.) as nucleic acid aptamers specific to prion, influenza viruses (H1N1, H3N2, H5N1, H9N2, etc.), human immunodeficiency viruses, hepatitis B viruses, hepatitis C viruses, bovine viral diarrhea viruses, vaccinia viruses, Zika viruses, RS viruses, herpes viruses, Japanese encephalitis viruses, cytomegaloviruses, rabies viruses, human papilloma viruses, Ebola viruses, noroviruses (GII, GII.3, GII.4, etc.), dengue viruses, or SARS coronaviruses (SARS-CoV). Examples of the nucleic acid aptamer specific to SARS coronavirus-2 (SARS-CoV-2) include CAGCACCGACCTTGTGCTTTGGGAGTGCTGGTCCAAGGGCGTTAATGGACA (SEQ ID NO: 6), and ATCCAGAGTGACGCAGCATTTCATCGGGTCCAAAAGGGGCTGCTCGGGATTG CGGATATGGACACGT (SEQ ID NO: 7) reported, for example, in Song Y, Song J, Wei X, Huang M, Sun M, Zhu L, Lin B, Shen H, Zhu Z, Yang C, Discovery of Aptamers Targeting Receptor-Binding Domain of the SARS-CoV-2 Spike Glycoprotein, Preprint from ChemRxiv, 4 Apr. 2020.

[2-3. Signal Substance]

The signal substance 52d functions to read out the binding information between the detection target and the nucleic acid aptamer 55d specific to the detection target. The signal substance 52d is not particularly limited as long as the signal intensity detected by the binding of the detection target to the concave 31 changes or the spectrum changes (for example, the peak shifts). Examples of the signal substance 52d include fluorescent substances, radioactive element-containing substances, and magnetic substances. From the viewpoint of easy detection and the like, the signal substance is preferably a fluorescent substance. Examples of the fluorescent substance include fluorescent dyes such as fluorescein dyes, cyanine dyes such as indocyanine dyes, and rhodamine dyes; fluorescent proteins such as GFP; and nanoparticles such as gold colloids and quantum dots. Examples of the radioactive element-containing substance include sugar, amino acids and nucleic acids labeled with a radioisotope such as ¹⁸F, and MRI probes labeled with ¹⁹F. Examples of the magnetic substance include those having a magnetic body such as ferrichrome and those found in ferrite nanoparticles, nanomagnetic particles and the like.

Further, the signal substance 52d can be configured as one of the fluorescent dye pair that causes fluorescence resonance energy transfer (FRET). The fluorescent dye pair that causes FRET is not particularly limited, and it is not limited whether a donor dye or an acceptor dye is selected as the signal substance 52d. Preferably, a donor dye can be selected as the signal substance 52d. Specific examples of the donor dye/acceptor dye constituting the fluorescent dye pair that causes FRET include fluorescein isothiocyanate (FITC)/tetramethylrhodamine isothiocyanate (TRITC), Alexa Fluor647/Cy5.5, HiLyte Fluor647/Cy5.5, and R-phycoerythrin (R-PE)/allophycocyanin (APC).

[3. Manufacture Method of Base Material for Producing Sensor for Analysis of Detection Target]

The manufacture method of a base material for producing a sensor for analysis of a detection target according to the present invention includes the following steps:

a monomolecular film formation step (1-1) of forming on a base material a monomolecular film having a polynucleotide group for nucleic acid aptamer's binding and a polymerization initiating group on a surface thereof;

a template introduction step (1-2) of introducing a template having on a surface thereof a polynucleotide group capable of complementary binding with the polynucleotide group for nucleic acid aptamer's binding into the polynucleotide group for nucleic acid aptamer's binding;

a surface modification step (1-3) of modifying a surface of the template with a polymerizable functional group via a reversible linked group;

a polymerization step (1-4) of forming a polymer film on a surface of the base material by adding a polymerizable monomer and synthesizing a molecularly imprinted polymer corresponding to a part of the surface of the template using the polymerizable monomer and the polymerizable functional group as substrates and the polymerization initiating group as a polymerization initiator; and

a removal step (1-5) of cleaving the complementary binding and the reversible linked group to convert the complementary binding and the reversible linked group into a polynucleotide group for nucleic acid aptamer's binding and a group for signal substance's binding, respectively, and removing the template.

FIGS. 3, 4, 5, 6, and 7 schematically show the monomolecular film formation step (1-1), template introduction step (1-2), surface modification step (1-3), polymerization step (1-4), and removal step (1-5), respectively. That is, in the illustrated aspects, the method of manufacturing the base material for producing a sensor for analysis 10 includes the following steps:

a monomolecular film formation step (1-1) of forming on a base material 20 a monomolecular film 21 having a polynucleotide group for nucleic acid aptamer's binding 25b and a polymerization initiating group 23a (FIG. 3);

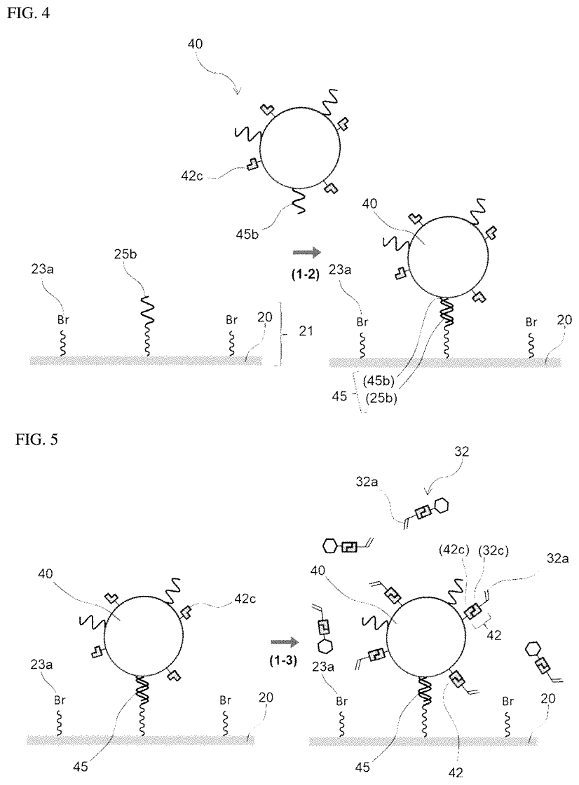

a template introduction step (1-2) of introducing a template 40 having on a surface thereof a polynucleotide group 45b capable of complementary binding 45 with the polynucleotide group for nucleic acid aptamer's binding 25b into the polynucleotide group for nucleic acid aptamer's binding 25b (FIG. 4);

a surface modification step (1-3) of modifying a surface of the template 40 with a polymerizable functional group 32a via a reversible linked group 42 (FIG. 5);

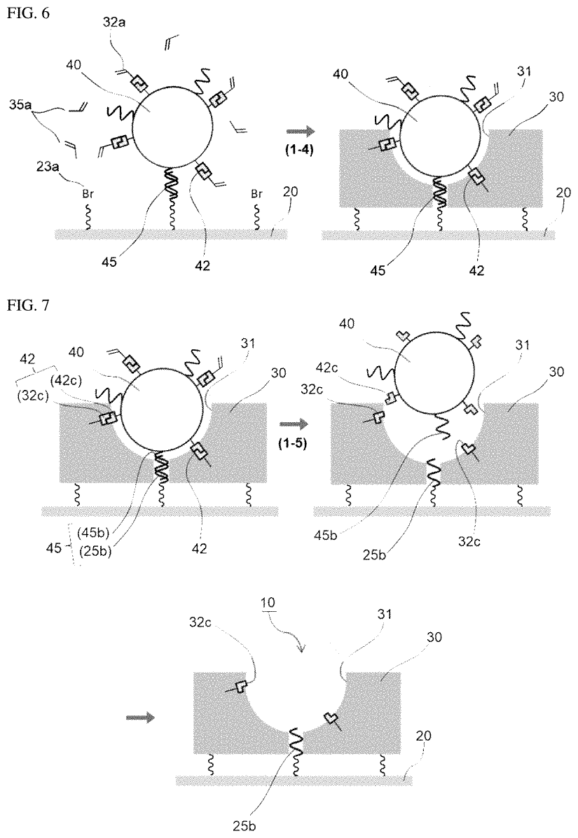

a polymerization step (1-4) of forming a polymer film 30 on a surface of the base material by adding a polymerizable monomer 35a and synthesizing a molecularly imprinted polymer corresponding to a part of the surface of the template 40 using the polymerizable monomer 35a and the polymerizable functional group 32a as substrates and the polymerization initiating group 23a as a polymerization initiator (FIG. 6); and

a removal step (1-5) of cleaving the complementary binding 45 and the reversible linked group 42 to convert the complementary binding 45 and the reversible linked group 42 into a polynucleotide group for nucleic acid aptamer's binding 25b and a group for signal substance's binding 32c, respectively, and removing the template 40 (FIG. 7).

Hereinafter, the respective steps will be described in detail with reference to the drawings.

[3-1. Monomolecular Film Formation Step]

As shown in FIG. 3, in the monomolecular film formation step, the monomolecular film 21 having the polynucleotide group for nucleic acid aptamer's binding 25b and the polymerization initiating group 23a on a surface thereof is formed on the base material 20.

In the illustrated example, specifically, a monomolecular film having a polymerization initiating group 23a and a binding functional group 25a on a surface thereof is firstly formed on a surface of the base material 20. The monomolecular film can be formed as a mixed self-assembled monomolecular film (mixed SAMs) by mixed self-assembly using a molecule having a polymerization initiating group 23a at the end, and a molecule having a binding functional group 25a which is different from the polymerization initiating group 23a at the end.

The polymerization initiating group 23a is not particularly limited as long as it has a structure capable of functioning as a polymerization initiator, and can be appropriately determined by those skilled in the art according to the polymerization reaction used in the polymerization step which will be described below. For example, examples of the polymerization-initiating group 23a include a group having a structure that generates a radical during a polymerization reaction, specifically, a carbon-halogen binding group (—CX group; X represents a halogen atom) derived from an organic halogen. In the illustrated aspect, the case where the polymerization initiating group 23a is a —CBr group is exemplified.

The binding functional group 25a is not particularly limited as long as it is a group capable of binding a polynucleotide 25, and can be appropriately determined by those skilled in the art. The polynucleotide 25 has a polynucleotide group for nucleic acid aptamer's binding 25b and a binding functional group 25c. The illustrated aspect exemplifies the case where the binding functional group 25a of the monomolecular film is a carboxyl group and the binding functional group 25c of the polynucleotide 25 is an amino group.

Next, the binding functional group 25a that is a carboxyl group in the monomolecular film is subjected to active esterification according to need, and then the binding functional group 25c of the polynucleotide 25 is reacted, so that the polynucleotide group for nucleic acid aptamer's binding 25b is extended. Thus, the monomolecular film 21 can be obtained.

[3-2. Template Introduction Step]

As shown in FIG. 4, in the template introduction step, the template 40 having on a surface thereof the polynucleotide group 45b capable of the complementary binding 45 with the polynucleotide group for nucleic acid aptamer's binding 25b is introduced into the polynucleotide group for nucleic acid aptamer's binding 25b on the surface of the monomolecular film 21.

The template 40 may be the same substance as the detection target, or may be a substance different from the detection target. In the present invention, an artificial particle can be used as the template 40. Since the artificial particle is an industrial product and controlled in particle size, size control and homogenization of the concave formed in the base material for producing a sensor for analysis are easy, and thus a sensor for analysis further excellent in analytical properties can be produced from the obtained base material for producing a sensor for analysis. So, the artificial particle is preferred in this regard.

The artificial particle used as the template 40 is not particularly limited as long as it can be used as a template in molecular imprinting, and includes artificially manufactured inorganic particles and organic particles. Examples of the inorganic particle include metals, oxides, nitrides, fluorides, sulfides and borides of metals, composite compounds thereof, and hydroxyapatite, and preferably include silicon dioxide (silica). Examples of the organic particle include latex cured products, dextran, chitosan, polylactic acid, poly(meth)acrylic acid, polystyrene, and polyethyleneimine.

Since the size of the concave 31 (see FIG. 1 and the like) depends on the size of the template 40, the size of the template 40 can be appropriately determined according to the size of the detection target. In order to form the concave 31 for receiving the detection target of interest, the template 40 having a size equal to or larger than that of the detection target can be used. For example, the average particle size of the template 40 particle is, for example, 1 nm to 10 μm, preferably 50 to 1 μm, more preferably 100 to 500 nm, further preferably 150 to 200 nm. The average particle size refers to a Z average particle size measured by the dynamic light scattering method.

The template 40 has on a surface thereof the polynucleotide group 45b capable of forming the complementary binding 45 with the polynucleotide group for nucleic acid aptamer's binding 25b. Specifically, assuming that the sequence of the polynucleotide group for nucleic acid aptamer's binding 25b is A, the polynucleotide group 45b of the template 40 is most preferably a completely complementary sequence consisting only of bases complementary to the sequence A. However, it also allows the inclusion of mismatched bases, as long as the complementary binding 45 can be formed.

In the illustrated aspect, the template 40 has a reversible binding group 42c on the surface thereof. The reversible binding group 42c is a group capable of forming the reversible linked group 42 (FIG. 5 which will be described below) by binding to the group for signal substance's binding 32c (FIG. 1 described above and FIG. 5 which will be described below). Examples of such a group include a thiol group (the corresponding reversible linked group 42 is a disulfide group), an aminooxy group or a carbonyl group (the corresponding reversible linked group 42 is an oxime group), a boronic acid group and a cis-diol group (the corresponding reversible linked group 42 is a cyclic diester group), an amino group and a carbonyl group (the corresponding reversible linked group 42 is a Schiff base), and an aldehyde group or ketone group and alcohol (the corresponding reversible linked group 42 is an acetal group). A thiol group is preferred.

The method of modifying the surface of the particle with a specific group is widely known, and thus those skilled in the art can appropriately introduce the polynucleotide group 45b and the reversible binding group 42c to be introduced based on a known surface modification method, considering the kinds of those groups and components of the artificial particle.

By binding the template 40 having the polynucleotide group 45b and the reversible binding group 42c on the surface thereof in this way, the complementary binding 45 is formed with respect to the polynucleotide group for nucleic acid aptamer's binding 25b on the base material 20, so that the template 40 is introduced.

In the schematic diagram given in the drawing, for the sake of convenience, one complementary binding 45 formed between the base material 20 and the template 40 is shown, but, actually, a plurality of complementary bindings 45 are formed between the base material 20 and the template 40.

[3-3. Surface Modification Step]

As shown in FIG. 5, in the surface modification step, the surface of the template 40 is modified with the polymerizable functional group 32a via the reversible linked group 42.

The reversible binding group 42c has only to be converted into the reversible linked group 42 by binding to any other reversible binding group (specifically, corresponding to the group for signal substance's binding 32c described above). Examples of the reversible binding group 42c include a thiol group (the corresponding reversible linked group 42 is a disulfide group), an aminooxy group or a carbonyl group (the corresponding reversible linked group 42 is an oxime group), a boronic acid group and a cis-diol group (the corresponding reversible linked group 42 is a cyclic diester group), an amino group and a carbonyl group (the corresponding reversible linked group 42 is a Schiff base), and an aldehyde group or ketone group and alcohol (the corresponding reversible linked group 42 is an acetal group).

The polymerizable functional group 32a has only to have a polymerizable unsaturated bond, and typical examples thereof include a (meth)acrylic group.

The illustrated aspect exemplifies an aspect in which a molecule 32 containing a (meth)acrylic group which is an example of the polymerizable functional group 32a and a disulfide bond is disulfide-exchanged to a thiol group which is an example of the reversible binding group 42c on the surface of the template 40 to convert the thiol group into the disulfide group which is the reversible linked group 42, thereby modifying the surface of the template 40 with the polymerizable functional group 32a.

Thus, by using the template 40 having the reversible binding group 42c on the surface thereof in advance, the reversible linked group 42 can be delivered only to the surface of the template 40.

[3-4. Polymerization Step]

As shown in FIG. 6, in the polymerization step, the polymerizable monomer 35a is added, and a molecularly imprinted polymer corresponding to a part of the surface of the template 40 is synthesized using the polymerizable monomer 35a and the polymerizable functional group 32a as substrates and the polymerization initiating group 23a as a polymerization initiator. As a result, the polymer film 30 having the concave 31 is formed on the surface of the base material 20. In the present specification, the polymer synthesized by imprinting polymerization using the template is referred to as molecularly imprinted polymer for the sake of convenience. In the present invention, a template that is not a molecule (for example, a microparticle having a membrane structure) is also allowed, so polymers synthesized by imprinting polymerization using a template that is not a molecule are also included in the molecularly imprinted polymer.

The polymerizable monomer 35 is a biocompatible monomer, preferably a hydrophilic monomer, more preferably a zwitterionic monomer, as described for the polymer film 30 above.

A zwitterionic monomer contains both an anionic group derived from an acidic functional group (for example, a phosphoric acid group, a sulfuric acid group, and a carboxyl group) and a cationic group derived front a basic functional group (for example, a primary amino group, a secondary amino group, a tertiary amino group, and a quaternary ammonium group) in one molecule. Examples of the zwitterionic monomer include phosphobetaine, sulfobetaine, and carboxybetaine.

More specifically, examples of the phosphobetaine include a molecule having a phosphorylcholine group in the side chain, and preferably include 2-methacryloyloxyethyl phosphorylcholine (MPC).

Examples of the sulfobetaine include N,N-dimethyl-N-(3-sulfopropyl)-3′-methacryloylaminopropaneaminium inner salt (SPB) and N,N-dimethyl-N-(4-sulfobutyl)-3′-methacryloylaminopropaneaminium inner salt (SBB).

Examples of the carboxybetaine include N,N-dimethyl-N-(1-carboxymethyl)-1-methacryloyloxyethaneaminium inner salt (CMB) and N,N-dimethyl-N-(2-carboxyethyl)-2′-methacryloyloxyethaneaminium inner salt (CEB).

By forming, on the surface of the base material 20, a polymerization reaction system in which the polymerizable functional group 32a, the polymerizable monomer 35a, the polymerization initiating group 23a and the template 40 coexist, surface-initiated atom transfer radical polymerization (SI-ATRP) progresses. The polymerization reaction system preferably further contains, as a polymerization catalyst, a transition metal or a transition metal complex formed from a transition metal compound and a ligand, and more preferably further uses a reducing agent.

Examples of the transition metal or transition metal compound include metallic copper or copper compounds, and examples of the copper compound include chloride, bromide, iodide, cyanide, oxide, hydroxide, acetate, sulfate, and nitric oxide, and preferably bromide. The ligand is preferably a polydentate amine, and specific examples thereof include bidentate to hexadentate ligands. Among these, bidentate ligands are preferred, 2,2-bipyridyl, 4,4′-di-(5-nonyl)-2,2′-bipyridyl, N-(n-propyl) pyridylmethanimine, N-(n-octyl)pyridylmethanimine and the like are more preferred, and 2,2-bipyridyl is further preferred.

Examples of the reducing agent include alcohols, aldehydes, phenols and organic acid compounds, and preferably organic acid compounds. Examples of the organic compound include citric acid, oxalic acid, ascorbic acid, ascorbic acid salts, and ascorbic acid esters, preferably ascorbic acid, ascorbic acid salts, and ascorbic acid esters, and more preferably ascorbic acid.

A polymer chain extends from the polymerization initiating group 23a, which is a radical generation source, using the polymerizable monomer 35a as a substrate, and the thickness of the polymer film increases. Also, the extending polymer chain incorporates the polymerizable functional group 32a modifying the surface of the template 40 as a substrate when reaching the surface of the template 40. Thus, a polymer is synthesized so that the concave 31 having a shape that conforms to the surface shape of the template 40 is formed. The polymer film can be grown to a thickness corresponding to about ½ to ⅓ of the diameter from the top to the bottom of the template 40 (when the upper side of the drawing is regarded as top) introduced into the base material 20. As a result, the polymer film 30 is obtained. As a reaction solvent in the polymerization reaction system, an aqueous solvent such as a buffer solution is preferably used.

[3-5. Removal Step]

As shown in FIG. 7, in the removal step, the complementary binding 45 is cleaved to be converted into the polynucleotide group for nucleic acid aptamer's binding 25b, and the reversible linked group 42 is cleaved to be converted into the group for signal substance's binding 32c, so that the template 40 is removed. The reversible linked group 42 is delivered only to the surface of the template 40 in the surface modification step above. Therefore, in the concave 31 of the polymer film 30, which is the trace of the removed template 40, the polynucleotide group for nucleic acid aptamer's binding 25b remains, and the group for signal substance's binding 32c produced from the reversible linked group 42 is placed only inside the concave 31. As a result, the base material for producing a sensor for analysis 10 is obtained.

In the illustrated schematic diagram, for the sake of convenience, one polynucleotide group for nucleic acid aptamer's binding 25b is formed in one concave 31, but, as described above, a plurality of complementary bindings 45 are formed between the base material 20 and the template 40, and thus a plurality of polynucleotide groups for nucleic acid aptamer's binding 25b are actually formed in one concave 31.

[4. Manufacture of Sensor for Analysis of Detection Target]

The manufacture method of a sensor for analysis of a detection target according to the present invention includes the following steps:

a step (1) of performing the manufacture method of a base material for producing a sensor for analysis of a detection target;

a step (2) of binding a nucleic acid aptamer specific to a detection target to the polynucleotide group for nucleic acid aptamer's binding by complementary binding; and

a step (3) of binding a signal substance to the group for signal substance's binding.

Regarding the order of steps (2) and (3), either of the steps may be performed first, or both of the steps may be performed at the same time.

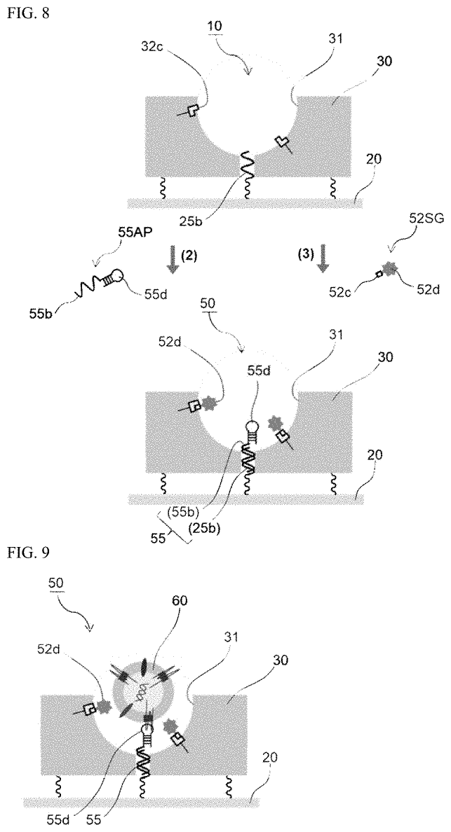

FIG. 8 schematically shows a manufacture method of a sensor for analysis of a detection target according to the present invention. That is, in the illustrated aspect, the manufacture method of the sensor for analysis of a detection target for analysis 50 includes the following steps:

a step (1) of performing the manufacture method of a base material for producing a sensor for analysis of a detection target 10;

a step (2) of binding a nucleic acid aptamer 55d specific to the detection target to the polynucleotide group for nucleic acid aptamer's binding 25b by complementary binding 55; and

a step (3) of binding a signal substance 52d to the group for signal substance's binding 32c.

Step (1) of manufacturing the base material for producing a sensor for analysis of a detection target 10 is as described in detail in the above “3. Manufacture method of base material for producing sensor for analysis of detection target”, and includes the above monomolecular film formation step (1-1), template introduction step (1-2), surface modification step (1-3), polymerization step (1-4) and removal step (1-5).

In step (2), as shown in FIG. 8, a component 55AP that gives the nucleic acid aptamer 55d is hybridized with the base material for producing a sensor for analysis of a detection target 10. The component 55AP that gives the nucleic acid aptamer 55d includes the nucleic acid aptamer 55d and the polynucleotide group 55b capable of forming the complementary binding 55 with the polynucleotide group for nucleic acid aptamer's binding 25b. The nucleic acid aptamer 55d is as described in the above “2-2. Nucleic acid aptamer (nucleic acid aptamer specific to detection target)”. Further, the polynucleotide group 55b is a polynucleotide extended to the nucleic acid aptamer 55d. The nucleic acid aptamer 55d and the polynucleotide group 55b may be directly bound, or the intervention of any other linked group (e.g., a base or a polynucleotide) is also allowed. The polynucleotide group 55b is similar to the group described as the polynucleotide group 45b of the template 40 in the above “3-2. Template introduction step”. Assuming that the sequence of the polynucleotide group for nucleic acid aptamer's binding 25b is A, the polynucleotide group 45b is most preferably a completely complementary sequence consisting only of bases complementary to the sequence A. However, it also allows the inclusion of mismatched bases, as long as the complementary binding 55 can be formed.

In the illustrated schematic diagram, for the sake of convenience, one nucleic acid aptamer 55d is introduced into one concave 31. However, as described above, a plurality of polynucleotide group for nucleic acid aptamer's binding 25b are present in one concave 31. Therefore, actually, a plurality of nucleic acid aptamers 55d are actually introduced into one concave 31.

In step (3), as shown in FIG. 8, a component 52SG that gives the signal substance 52d is reacted with the base material for producing a sensor for analysis of a detection target 10. The component 52SG that gives the signal substance 52d includes the signal substance 52d and a binding group 52c. The signal substance 52d is as described in the above “2-3. Signal substance”. As the binding group 52c, a group capable of reacting with and binding to the group for signal substance's binding 32c is selected.

Since the base material for producing a sensor for analysis 10 has the group for signal substance's binding 32c only in the concave 31 serving as a sensor field on the surface of the base plate 20, the signal substance 52d can be arranged only in the concave 31 due to the reactivity of the binding group 52c.

For one sensor for analysis production base material 10, one kind of nucleic acid aptamer 55d and one kind of signal substance 52d may be introduced into all the concaves 31. Or, one kind of nucleic acid aptamer 55d is introduced into one concave 31, another kind of nucleic acid aptamer 55d is introduced into another concave 31, and different kinds of signal substances may be introduced, corresponding to the respective kinds of nucleic acid aptamers.

[5. Method for Analyzing Target Substance]

FIG. 9 shows a schematic diagram illustrating an example of a method for analyzing a detection target according to the present invention. As shown in FIG. 9, in the method for analyzing a detection target of the present invention, an analytical sample liquid containing the detection target 60 is brought into contact with the surface of the base material 20 of the sensor for analysis 50.

The detection target 60 is not particularly limited in principle as long as it is a substance that specifically binds to the nucleic acid aptamer 55d, and examples thereof include the substances described in the above “2-1. Detection target”.

The aspect of the analytical sample liquid containing the detection target 60 is not particularly limited, but, from the viewpoint of the rapidity of analysis, it is preferable that the analytical sample liquid has not been subjected to the treatment for separating the detection target 60. Examples of the treatment for separating the detection target 60 include ultracentrifugation, ultrafiltration, continuous flow electrophoresis, filtration using a size filter, and gel filtration chromatography.

As the analytical sample liquid containing the detection target 60, a sample obtained from the environment in which the detection target 60 is present (when the detection target 60 is a cell or an extracellular vesicle), or a sample obtained from the environment in which the detection target 60 may occur (when the detection target 60 is an extracellular vesicle and is a product from a cell). Specifically, it may be a biological sample containing cells. When the detection target 60 is an extracellular vesicle such as a small extracellular vesicle, examples of cells producing the detection target 60 include cancer cells, mast cells, dendritic cells, reticulocytes, epithelial cells, B cells, and nerve cells. More specifically, examples of the analytical sample liquid containing the detection target 60 include body fluids such as blood, milk, urine, saliva, lymph, cerebrospinal fluid, amniotic fluid, tears, sweat, and rhinorrhea. Treatment liquids obtained by subjecting these body fluids to pretreatment such as removal of unnecessary components, and culture fluids obtained by culturing cells contained in these body fluids are also included in the analytical sample liquid. Of these analysis sample liquids, body fluids such as urine, saliva, tear fluid, sweat, and rhinorrhea are particularly preferred in terms of non-invasiveness and easy collection.

When the analytical sample liquid containing the detection target 60 is brought into contact with the surface of the base material 20 of the sensor for analysis 50, the detection target 60 is specifically captured by the nucleic acid aptamer 55d in the concave 31. For example, when the detection target 60 is a small extracellular vesicle, the small extracellular vesicle is captured by binding specifically to the nucleic acid aptamer 55d via CD63, CD9, CD81, CD37, CD53, CD82, CD13., CD11, CD86, ICAM-1, Rab5, Annexin V, LAMP1 or the like as a membrane protein (small extracellular vesicle-specific antigen). When the detection target 60 is a cancer cell, the cancer cell is captured by specifically binding to the nucleic acid aptamer 55d via Caveolin-1, EpCAM, FasL, TRAIL, Galectine3, CD151, Tetraspanin 8, EGFR, HER2, RPN2, CD44, TGF-β or the like as a cancer cell-specific antigen.

When the detection target 60 is specifically captured by the nucleic acid aptamer 55d in the concave 31, the signal substance 52d undergoes an environmental change by the detection target 60 at that moment, so that a signal change is caused before and after the detection target 60 is captured. That is, the sensor for analysis 50 can read the binding information of the detection target 60 that serves as a sensing target by a signal change, and the detection target 60 is detected by this signal change. Since the capture of the detection target 60 and the signal change occur almost at the same time, the detection can be performed rapidly without need to add a reagent for detecting the detection target 60.

In the case where the sensor for analysis 50 is configured so that the signal substance 52d is one of the fluorescent dye pair that causes fluorescence resonance energy transfer (FRET), and the other of the fluorescent dye pair is bound to the detection target 60 in advance, when the detection target 60 is specifically captured by the nucleic acid aptamer 55d in the concave 31, the fluorescent dye in the signal substance 52d and the fluorescent dye in the detection target 60 are close to each other at that moment, so that fluorescence is emitted by FRET. The detection target 60 is detected by the fluorescence emission by this FRET. Since the capture of the detection target 60 and the fluorescence emission by FRET occur almost at the same time, the detection can be performed rapidly without need to add a reagent for detecting the detection target 60. The fluorescent dye pair that causes FRET is not particularly limited, and it is not limited whether a donor dye or an acceptor dye is selected as the signal substance 52d. Preferably, a donor dye can be selected as the signal substance 52d. Specific examples of the donor dye/acceptor dye constituting the fluorescent dye pair that causes FRET include fluorescein isothiocyanate (FITC)/tetramethylrhodamine isothiocyanate (TRITC), Alexa Fluor647/Cy5.5, HiLyte Fluor647/Cy5.5, and R-phycoerythrin (R-PE)/allophycocyanin (APC).

Further, since the sensor for analysis 50 of the present invention has substantially no signal substance 52d in a portion other than the concave 31 on the surface of the base material 20, the sensor is not affected by an undesired background even if there is nonspecific adsorption in a portion outside the concave 31 on the surface of the base material 20. Therefore, the detection target 60 can be detected with high sensitivity.

For the sake of convenience, the schematic diagram given in the drawing merely shows that one of the molecules expressed on the surface of the detection target 60 in one concave 31 is specifically captured by the nucleic acid aptamer 55d. However, a plurality of the molecules are expressed on the surface of the detection target 60, and, as described above, a plurality of nucleic acid aptamers 55d are introduced into one concave 31. So, the plurality of molecules expressed on the surface of the detection target 60 are specifically captured by the plurality of nucleic acid aptamers 55d. Therefore, the affinity of the nucleic acid aptamer 55d per the detection target 60 is raised to a surprising level.

### EXAMPLES

Hereinafter, the present invention will be described in detail based on examples, but is not restricted by these examples.

### Example 1: Manufacture of Base Material for Producing Sensor for Analysis of Detection Target

In this example, the specific example of the base material for producing a sensor for analysis of a detection target shown in FIG. 1 was manufactured based on the specific example of the manufacture method of a base material for producing a sensor for analysis of a detection target shown in FIGS. 3 to 7.

(Reagents and the Like)
Artificial particles as a material for the template 40: silica particles; sicastar (registered trademark)-redF; silica particles having a carboxyl group on surfaces thereof, Z average particle size measured by the dynamic light scattering method with Zetasizer Nano ZS MAL500735 manufactured by Malvern being 205 nm (pdi: 0.017)
Polynucleotide group 45b: ODN2; CACAAATCTGTCGCTGAGTA (SEQ ID NO: 8)
Reagent that gives the polynucleotide group 45b: ODN2-NH₂; a molecule in which an amino group was added to the 3′ end of ODN2
Reversible binding group 42c: thiol group
Reagent that gives the reversible binding group 42c: 2-aminoethanethiol hydrochloride
Base plate 20: gold-sputtered base plate
Binding functional group 25a: carboxyl group
Molecule having the binding functional group 25a at the end: 11-mercapto-undecanoic acid
Polymerization initiating group 23a: organic bromo group
Molecule having the polymerization initiating group 23a at the end: 2-(2-bromoisobutyryloxy)-undecyl thiol
Polynucleotide group for nucleic acid aptamer's binding 25b: ODN1; TACTCAGCGACAGATTTGTG (SEQ ID NO: 9)
Binding functional group 25c: amino group
Polynucleotide 25:ODN1-NH₂; a molecule in which an amino group was added to the 3′ end of ODN1
Polymerizable functional group 32a: acryloyl group
Molecule 32 containing the polymerizable functional group 32a and a disulfide bond: 2-(2-pyridyldithio)ethyl acryloylamide
Polymerizable monomer 35a: 2-methacryloyloxyethyl phosphorylcholine (MPC)

(1) Production of Template 40

In 1 mL of a suspension containing silica particles (COOH: 25 nmol) in water, an aqueous solution of 100 μM ODN2-NH₂, and an aqueous solution of 0.10 mM 2-aminoethanethiol hydrochloride (2.5 nmol) were mixed. Then, an aqueous solution of 0.10 mM DMT-MM (25 nmol) was added, and the mixture was reacted at 25° C. with stirring overnight. Then, the particles were purified by performing a series of operations of centrifugation (10000 G, 10 minutes) and replacement of the supernatant with pure water three times. As a result, a template having a thiol group and ODN2 introduced onto the surface of the silica particles was obtained.

(2) Production of monomolecular film 21 (step 1-1)

After washing with ethanol and spraying with nitrogen, a gold-sputtered glass base plate subjected to UV ozone treatment for 20 minutes was immersed in 1 mL of an ethanol solution containing 0.30 mM 11-mercapto-undecanoic acid and 0.60 mM 2-(2-bromoisobutyryloxy)-undecyl thiol at 25° C. overnight. The base plate after the reaction was washed with ethanol and dried by spraying nitrogen. As a result, a monomolecular film having a carboxyl group and a polymerization initiating group was obtained on the surface of the base plate.

In 3 mL of dry dichloromethane, 5.0×10⁻² μmol of 1-ethyl-3-(3-dimethylaminopropyl) carbodiimide hydrochloride, 5.0×10⁻² μmol of N-hydroxysuccinimide, and 5.0×10⁻² μmol of N,N-diisopropylethylamine were dissolved, and the base plate on which the obtained monomolecular film was formed was immersed in this solution at 25° C. overnight to perform active esterification of the binding functional group 25a (carboxyl group) on the surface of the monomolecular film. After washing with dry dichloromethane, a 40 μl of a solution of 10 μM ODN1-NH₂ (10 mM PBS (pH 7.4) and 100 mM NaCl) was added dropwise, and the mixture was reacted at 25° C. for 3 hours to introduce ODN1. As a result, a monomolecular film (monomolecular film 21) having ODN1 and a polymerization initiating group was formed on the surface of the base plate.

(3) Introduction of Template 40 (Step 1-2)

The base plate on which the monomolecular film 21 produced in the above (2) was formed was set on a dip coater and immersed in 3.5 mL of an aqueous solution of 1 mg/mL the template 40 produced in (1) for 30 minutes. After pulling up the base plate at 1 mm/min, the base material was placed in a PCR tube filled with 250 μl of PBS, heated at 60° C. for 10 minutes using a thermal cycler (manufactured by Takara Bio, TaKaRa Thermal Cycler Dice Touch, the same below), and cooled to 25° C. over 30 minutes, to hybridize the polynucleotide group 45b (ODN2) of the template 40 with the polynucleotide group for nucleic acid aptamer's binding 25b (ODN1) on the base plate. Thus, the template was introduced onto the base plate.

(4) Modification of Surface of Template 40 (Step 1-3)

The base plate onto which the template 40 was introduced was immersed in 1 mL of a PBS (pH.7.4) solution of 100 μM 2-(2-pyridyldithio)ethyl acrylamide, and a reaction was caused overnight at 25° C. to carry out a disulfide exchange reaction. Thus, the template surface was modified with acryloyl groups.

(5) Synthesis of Polymer Film (Step 1-4)

A prepolymer solution obtained by dissolving 50 mM MPC, 1 mM CuBr₂, and 2 mM 2,2′-bipyridyl in 9 mL of 10 mM PBS (pH 7.4, 100 mM NaCl), and the base plate obtained in the above (4) were placed in a Schlenk flask. The flask was sealed with a silicon stopper, and deaerated and replaced with nitrogen. Using a disposable syringe, 1 mL of a solution of 0.5 mM L-ascorbic acid (10 mM PBS (pH7.4)) was added to the prepolymer solution in the Schlenk flask. Further, by deaeration and nitrogen replacement, and shaking in a water bath at 40° C. for 3 hours, surface-initiated atom transfer radical polymerization (SI-ATRP) was performed. The base plate after the polymerization was washed with pure water, dried by spraying nitrogen, and then immersed in 1 mL of an aqueous solution of 100 mM EDTA-4Na at 25° C. for 15 minutes to remove copper ions remaining inside the polymer film. Thus, a polymer film was synthesized on the surface of the base plate.

(6) Removal of Template (Step 1-5)

The polymer film base plate obtained in the above (5) was placed in a PCR tube, immersed in 250 μl of an aqueous solution of 50 mM tris(2-carboxyethyl)phosphine, and heated in a thermal cycler at 60° C. for 3 hours. A disulfide bond was reduced and converted to a thiol group, the base plate was washed with pure water, immersed in 250 μl of an aqueous solution of 2M urea, and heated at 99° C. for 30 minutes in a thermal cycler to cleave complementary binding and convert it to ODN1. Then, the base plate was washed with pure water. Thus, the template was removed. By the above operations, the base material for producing a sensor for analysis of a detection target having a thiol group and ODN1 in the concave by molecular imprinting of the template provided on the polymer film on the base plate was obtained.

### Example 2: Production of Sensor for Analysis of Small Extracellular Vesicle CD63 Expressed on the Surface

In this example, as the specific example of the sensor for analysis of a detection target shown in FIG. 2, a sensor for analysis of a small extracellular vesicle CD63 was manufactured based on the specific example of the manufacture method of a sensor for analysis of a detection target shown in FIG. 8.

(Reagents and the Like)
Component 55AP that gives the nucleic acid aptamer 55d: DNA aptamer-containing polynucleotide; ODN was extended at the 5′ end of the CD63-specific DNA aptamer;

### CACAAATCTGTCGCTGAGTACACCCCACCTCGCTCCCGTGACACTAATGCTA (SEQ ID NO: 10)

Nucleic acid aptamer 55d: CD63-specific DNA aptamer; CACCCCACCTCGCTCCCGTGACACTAATGCTA (SEQ ID NO: 1)
Polynucleotide group 55b: ODN2; CACAAATCTGTCGCTGAGTA (SEQ ID NO: 6)
Component 52SG that gives the signal substance 52d: Alexa Fluor (registered trademark) 647C 2 Maleimide
Signal substance 52d: fluorescent substance; Alexa Fluor (registered trademark) 647
Binding group 52c: maleimide group

(1) Introduction of CD63-Specific DNA Aptamer (Step 2)

A solution (250 μl) prepared by adjusting the DNA aptamer-containing polynucleotide to 1 μM (10 mM PBS, pH 7.4, 100 mM NaCl) was annealed (95° C., 10 minutes, and then cooled to 25° C. over 30 minutes). The base material for producing a sensor for analysis of a detection target obtained in Example 1 was placed in a PCR tube and filled with the annealed DNA aptamer-containing polynucleotide solution. In a thermal cycler, the base material was heated at 60° C. for 10 minutes and cooled to 25° C. over 30 minutes to hybridize ODN1 on the base plate with ODN2 of the DNA aptamer-containing polynucleotide. As a result, the CD63-specific DNA aptamer was introduced into the base material for producing a sensor for analysis.

(2) Introduction of Fluorescent Substance

Onto the base plate into which the CD63-specific DNA aptamer was introduced in the above (1), 40 μl of a solution of 100 μM Alexa Fluor (registered trademark) 647 C2 Maleimide (10 mM PBS pH7.4, 100 mM NaCl) was added dropwise, so that a Michael addition reaction was performed at room temperature for 1 hour. As a result, a fluorescent substance was introduced into the base material for producing a sensor for analysis. By the above operations, a sensor for analysis of small extracellular vesicles CD63 having a fluorescent substance and a CD63-specific DNA aptamer in the concave formed by molecular imprinting of the template provided on the polymer film on the base plate was obtained.

### Example 3: Method for Analyzing Small Extracellular Vesicle CD63 Using Sensor for Analyzing Small Extracellular Vesicle CD63

In this experimental example, the sensor for analysis of small extracellular vesicles CD63 obtained in Example 2 was used for analysis of sEVs obtained from the culture supernatant of a human prostate cancer PC3 cell line (manufactured by HNB, HBM PC3 100, PC3-derived sEVs) as an analysis target.

As a solution to be analyzed, sEVs derived from PC3 were prepared in PBS so as to attain 0, 0.01, 0.05, 0.1, 0.5, 1, 5, and 10 ng/mL. To the base plate for the sensor for analysis of small extracellular vesicles CD63, 40 μl of the solution to be analyzed was added dropwise, followed by a reaction at 25° C. for 1 minute, washing with 1 mL of PBS, and fluorescence measurement under the following conditions. The ROI for fluorescence measurement was taken for each bright spot, and 30 ROIs were obtained for each solution to be analyzed. The measured value was defined as average value of fluorescence intensity.

(Fluorescence Measurement Conditions)
Fluorescent microscope: IX73 inverted microscope manufactured by Olympus
Filter: CY5-4040C (604 to 644 nm for excitation and 672 to 712 nm for emission) manufactured by Olympus
Objective lens: ×100; UPLSAPO100XO manufactured by Olympus
Amount of light: 100%
Exposure time: 0.1 seconds
Light source: water source lamp; HGLGPS-SET manufactured by Olympus

When an adsorption isotherm was created by measuring the change in fluorescence intensity with respect to the small extracellular vesicle concentration (Io-I)/Io), significant fluorescence quenching was confirmed by the specific adsorption of the PC3-derived sEVs, and a specific response was observed.

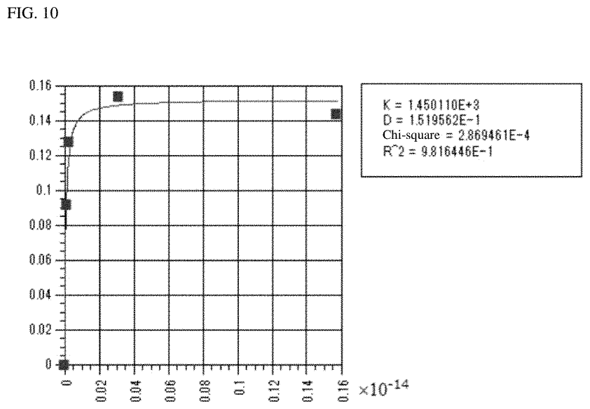

FIG. 10 shows the result of calculating the dissociation constant by curve fitting (regression analysis) of the obtained adsorption isotherm. As software, the analysis software DeltaGraph 5.4.5 v manufactured by Nihon Poladigital, K.K. was used, and fitting was performed based on the following formula. In the formula, Ka represents the binding constant, Kd represents the dissociation constant, G represents the small extracellular vesicle concentration, H is calculated from the fitting curve, and D represents the maximum change in fluorescence change rate.

$$[ Mathematical Formula 1 ]$$

$$Y = [ ( 1 + {K}_{a} G + {K}_{a} H ) - \sqrt{( 1 + {K}_{a} G + {K}_{a} H ) - 4 {AKK}_{a}^{2} HG ]} \times \frac{D}{2 {K}_{a} H} \\ {K}_{d} = 1 / {K}_{a}$$

Furthermore, the limit of detection (LOD) was obtained based on the following formula. In the formula, m represents the slope of a linear region of the adsorption isotherm (concentration range: 0, 0.01, 0.05, and 0.1 ng/mL), and S_{D} represents the standard deviation at the concentration of 0 ng/mL.

LOD=3.3S_{D}/m [Mathematical Formula 2]

As a result, the dissociation constant Kd was calculated to be 6.9×10⁻¹⁸ [M], indicating a high binding ability. The LOD was calculated to be 0.16 ng/mL. Since the sEVs used this time were 1.90×10¹¹ particles/mg, the LOD was 2.95×10⁵ particles/mL when converted to the number of sEVs. This value was significantly lower than the number of sEVs in the blood, 10¹¹ particles/mL, and the number of sEVs in body fluids, 10⁸ to 10¹¹ particles/mL. Further, as shown in “CD63 Aptamer Data Sheet”, [online], Apr. 1, 1998, BasePair Biotechnologies, Inc., [Searched: Mar. 25, 2020] Internet <URL: https://www.basepairbio.com/wp-content/uploads/2017/04/ATW0056-CD63-Aptamer-Data-Sheet_15 Sept17.pdf>, the dissociation constant Kd of the CD63 aptamer into the CD63 fragment is 17.1 n[M], that is, 10⁻⁸ [M] order. Furthermore, as will be presented in Comparative Example 2 below, the dissociation constant Kd is 3.8×10⁻¹⁵ [M] in the sensor for analysis of sEVs using an antibody instead of the nucleic acid aptamer. In other words, it was found that the sensing environment inside the minute concave formed by molecular imprinting exhibits an extremely high affinity when using the nucleic acid aptamer as compared with the case of using an antibody as the molecule specific to the detection target.

### Comparative Example 1: Method for Analyzing Small Extracellular Vesicle CD63 Using Sensor for Analysis of Small Extracellular Vesicle CD63 without CD63-Specific DNA Aptamer

A sensor for CD63 analysis (Comparative Example 1-1) was produced in the same manner as in Example 3 except that a random polynucleotide having no specificity to CD63 (TGTGCGGCGAAATATTATAGCTACCGCAATTA (SEQ ID NO: 11)) was used, instead of the CD63-specific DNA aptamer (SEQ ID NO: 1), as the nucleic acid to be introduced into ODN1 inside the concave on the base material for producing a sensor for analysis of a detection target. Further, a sensor for analysis of CD63 (Comparative Example 1-2) was produced in the same manner as in Example 3 except that nothing was introduced into ODN1 inside the concave on the base material for producing a sensor for analysis of a detection target.

For the sensors for the analysis of small extracellular vesicles CD63 having no CD63-specific DNA aptamer of Comparative Examples 1-1 and 1-2, PC3-derived sEVs were analyzed in the same manner as in Example 3 to create an adsorption isotherm. As a result, only slight fluorescence quenching due to nonspecific adsorption of PC-derived sEVs was confirmed, and no specific response was observed.

### Example 4: Manufacture of Sensor for Analysis of Small Extracellular Vesicle MUC-1 Expressed on the Surface

A sensor for analysis of MUC-1 was produced in the same manner as in Example 3 except that a MUC-1-specific DNA aptamer (SEQ ID NO: 2) was used, in place of the CD63-specific DNA aptamer (SEQ ID NO: 1), as the nucleic acid to be introduced into ODN1 in the concave on the base material for producing the sensor to be detected.

For the obtained sensor for analysis of MUC-1, sEVs were analyzed in the same manner as in Example 3 using, as analysis targets, human cancer cell line MCF-7-derived sEVs (manufactured by SB1, EXOP-100A-1, MUC-1 was expressed on the surface) and human healthy serum-derived sEVs (manufactured by SB1, EXOP-500A-1, MUC-1 was not expressed on the surface), respectively, to create an adsorption isotherm. As a result, when MCF-7-derived sEVs were used as the analysis target, significant fluorescence quenching due to specific adsorption of MCF-7-derived sEVs was confirmed, and a specific response was observed. On the other hand, no response was observed for the healthy human-derived sEVs.

### Comparative Example 2: Method for Analysis Using Antibody-Introduced Sensor for Analysis of Small Extracellular Vesicle

A sensor for analysis of sEVs, in which an antibody was introduced instead of the nucleic acid aptamer, was produced. Specifically, (i) a base material for producing a sensor for analysis of a detection target, including: a base material; and a polymer film provided on a surface of the base material, wherein the polymer film includes a concave that receives a detection target, and, inside the concave, a group for signal substance's binding and a group for antibody's binding was produced. Then, (ii) a sensor for analysis of a detection target was produced by introducing an antibody substance specific to the detection target into the group for antibody substance binding; and introducing a signal substance into the group for signal substance's binding.

(1) Synthesis of Template-Synthesis of Silica Nanoparticle into which Thiol Group and Histidine Tag (His-Tag) were Introduced

FITC-labeled silica nanoparticles (having 5 nmol of —COOH on the surface per 200 μl, a particle size of 200 nm) (200 μl) were dispersed in dichloromethane (DCM) (silica nanoparticle dispersion). 1-Ethyl-3-(3-dimethylaminopropyl)carbodiimide hydrochloride (EDC: 50 nmol, 10 eq), N-hydroxysuccinimide (NHS: 50 nmol, 10 eq), and N,N-diisopropylethylamine (DIEA: 50 nmol, 10 eq) were dissolved in dry DCM, and mixed with the silica nanoparticle dispersion. The mixture was reacted overnight to modify the surface of the silica nanoparticles with NHS. A His-tag with 6 histidines linked by peptide bond (having a lysine residue at the end and having a free ε-amino group: 0.10 μmol, 40 eq) and 2-aminoethanethiol hydrochloride (0.1 μmol, 40 eq) were added to the surface-modified silica nanoparticles, and reacted at room temperature. After completion of the reaction, silica nanoparticles into which a thiol group and His-tag were introduced (SH/His-tagged silica nanoparticles) were purified by centrifugation and filtration.

(2) Production of Base Material for Producing Sensor for Analysis of Detection Target in which Silica Nanoparticle was Used as Template

As will be described below, a mixed self-assembled monomolecular film (mixed SAMs) having an amino group and a bromo group at the end on a gold thin film deposited glass base plate was produced (molecular film formation step), an NTA group was introduced into the amino group at the end to form a NTA-Ni complex, then silica nanoparticles were immobilized by chelate binding (template introduction step). Then, the silica nanoparticles were modified with a methacrylic group (surface modification step), and a polymer thin film was synthesized by surface-initiated control/living radical polymerization (polymerization step). Finally, the silica nanoparticles were removed (removal step) to obtain a base material for producing a sensor for analysis of a detection target.

(2-1) Formation of Mixed Self-Assembled Monomolecular Film Having Bromo Group and Amino Group (Monomolecular Film Formation Step)

In the same manner as in Item 1 in Example 1, organic residues in the gold thin film deposited glass base plate were removed and the gold thin film deposited glass base plate was washed. The base plate was immersed in an ethanol solution of 0.5 mM amino-EG6-undecanthiol hydrochloride and 0.5 mM 2-(2-bromoisobutyryloxy)undecyl thiol and allowed to stand at 25° C. for 24 hours to form a mixed self-assembled monomolecular film having a bromo group and an amino group.

(2-2) Immobilization of SH/His-Tagged Silica Nanoparticle Via Ni-NTA (Template Introduction Step)

A DMSO solution (80 μL) of 5 mM isothiocyanobenzyl-nitrilotriacetic acid (ITC-NTA) was added dropwise to the base plate and allowed to stand at 25° C. for 2 hours to modify the amino group with NTA. After washing the base plate with DMSO and pure water, 100 μL of an aqueous solution of 4 mM NiCl₂ was added dropwise to the base plate and allowed to stand at room temperature for 15 minutes to form an Ni-NTA complex. Thereafter, 100 μl of an aqueous solution containing SH/His-tagged silica nanoparticles (solid content concentration: 5.1 mg/mi) was added dropwise to the base plate and allowed to stand at 25° C. for 1 hour.

(2-3) Methacryloylation of Immobilized SH/His-Tagged Silica Nanoparticle (Surface Modification Step)

By immersing the base plate in a PBS (pH 7.4) solution of 100 μM 2-(2-pyridyldithio)ethyl methacrylate and allowing it to stand overnight, the methacryloyl group was introduced into the SH group on the silica nanoparticle surface via disulfide by a disulfide exchange reaction.

(2-4) Production of MIP Thin Film (Polymerization Step)

A polymer thin film was synthesized on the base plate in the same manner as in item 4 of Example 1 except that the polymerization time was 3 hours. As a result, a polymer thin film in which the methacryloyl group of the silica nanoparticles was copolymerized together with the monomers was obtained on the base plate. After completion of the polymerization, the base plate was immersed in an aqueous solution of 1M ethylenediaminetetraacetic acid-4Na for 15 minutes to remove Cu²⁺ used for ATRP.

(2-5) Removal of Silica Nanoparticle (Removal Step)

The base plate was immersed in an aqueous solution of 50 mM tris(2-carboxyethyl)phosphine/HCl (TCEP) at 25° C. for 3 hours to reduce and cleave the disulfide bond binding the polymer to silica nanoparticles. Though a free SH group remains on the polymer side, this SH group is derived from SH/His-tagged silica nanoparticles, thus it is not present in a portion other than the concave corresponding to the template in the polymer thin film, and it is present only in the concave corresponding to the template. Though it is highly possible that, in the above-described EDTA-4Na treatment, the nickel in Ni-NTA was also removed and the His-tag became free at that time, the following operation was also performed as a precaution. The base plate was washed with pure water, then immersed in 50 mM acetate buffer (pH 4.0) containing 0.5 wt % SDS to wash out silica nanoparticles bound via Ni-NTA and His-tag from the polymer thin film.

(3) To form Ni-NTA again on the obtained base material for producing a sensor for analysis (MIP base plate), the MIP base plate was treated with an aqueous solution of 4 mM NiCl₂. Then, 100 μM His-tag Protein G dissolved in PBS was added to the base plate to immobilize Protein G capable of binding the antibody. Finally, 0.3 μM Anti-CD9 antibody dissolved in PBS was added to the base plate to immobilize the Anti-CD9 antibody via Protein G. Since Protein G binds to the Fc region of the antibody, the orientation of the immobilized antibody is uniform.

Further, a fluorescent molecule was selectively introduced into the concave in the sensor for analysis using thiol-reactive Alexa Fluor (registered trademark) 647 C2 Maleimide as the fluorescent molecule. The fluorescence intensity before introduction was 113±0.6 (n−3), whereas the fluorescence intensity after introduction was 151±2.1 (n−3), confirming the introduction of fluorescence. Thus, an antibody-introduced sensor for analysis of a detection target was obtained.

(4) Using the obtained antibody-introduced sensor for analysis, the binding behavior of sEVs was observed. As the analysis target, a PBS (10 mM phosphate, 140 mM NaCl, pH 7.4) solution of sEVs (concentrations of 0.01, 0.05, 0.1, 0.5, 1, 5, and 10 ng/mL, respectively) was used. Fluorescence detection of sEVs was performed under a microscope. The fluorescent microscope measurement conditions are as follows.

Filter: Cy5

Objective lens: ×5

Exposure time: 0.1 seconds

Light source: mercury lamp

The dissociation constant was calculated from the adsorption isotherm by the method described in Example 3. As a result, the dissociation constant was K_{d}=3.8×10⁻¹⁵ [M]

Although the preferred embodiments of the present invention are as described above, the present invention is not limited thereto, and various other embodiments can be made without departing from the spirit of the present invention.

### REFERENCE SIGNS LIST

10 Base material for producing sensor for analysis
20 Base material
21 Monomolecular film
25b Polynucleotide group for nucleic acid aptamer's binding (reversible binding group)
25a Binding functional group
23a Polymerization initiating group
30 Polymer film
31 Concave
32c Group for signal substance's binding (reversible binding group)
32a Polymerizable functional group
35a Polymerizable monomer
40 Template
42c Reversible binding group (reversible bond group capable of forming reversible linked group by binding to group for signal substance's binding)
45b Polynucleotide group
45 Complementary binding
50 Sensor for analysis
55d Nucleic acid aptamer specific to detection target
55 Complementary binding
52d Signal substance
60 Detection target

### SEQUENCE LIST FREE TEXT

SEQ ID NO: 1 is a DNA aptamer specific to CD63.
SEQ ID NO: 2 is a DNA aptamer specific to MUC-1.
SEQ ID NO: 3 is a DNA aptamer specific to EpCAM.
SEQ ID NO: 4 is a DNA aptamer specific to HER2.
SEQ ID NO: 5 is a DNA aptamer specific to ERa.
SEQ ID NO: 6 is a DNA specific to SARS coronavirus-2 (SARS-CoV-2).
SEQ ID NO: 7 is a DNA specific to SARS coronavirus-2 (SARS-CoV-2).
SEQ ID NO: 8 is a polynucleotide that can hybridize with a polynucleotide group for nucleic acid aptamer's binding.
SEQ ID NO: 9 is a sequence of a polynucleotide group for nucleic acid aptamer's binding.
SEQ ID NO: 10 is a polynucleotide that gives a nucleic acid aptamer.
SEQ ID NO: 11 is a random polynucleotide having no specificity to CD63.

What is claimed is:

1. A base material for producing a sensor for analysis of a detection target, comprising:
a base material; and
a polymer film provided on a surface of the base material,
wherein the polymer film includes a concave that receives a detection target, and, inside the concave, a group for signal substance's binding and a polynucleotide group for nucleic acid aptamer's binding.

2. The base material for producing a sensor for analysis of a detection target according to claim 1, wherein the polynucleotide group for nucleic acid aptamer's binding has a length of 8 bases or more.

3. The base material for producing a sensor for analysis of a detection target according to claim 1, wherein the polynucleotide group for nucleic acid aptamer's binding is a single chain.

4. The base material for producing a sensor for analysis of a detection target according to claim 1, wherein the polymer film is composed of a molecularly imprinted polymer prepared using the detection target or an object larger in size than the detection target as a template, and the concave corresponds to a part of a surface shape of the template.

5. The base material for producing a sensor for analysis of a detection target according to claim 1, wherein the group for signal substance's binding is a thiol group.

6. A sensor for analysis of a detection target comprising:
the base material for producing a sensor for analysis of a detection target according to claim 1;
a nucleic acid aptamer specific to the detection target, which is bound to the polynucleotide group for nucleic acid aptamer's binding; and
a signal substance which is bound to the group for signal substance's binding.

7. The sensor for analysis of a detection target according to claim 6, wherein the detection target is a microparticle having a membrane structure.

8. The sensor for analysis of a detection target according to claim 7, wherein the microparticle having a membrane structure is an extracellular vesicle.

9. The sensor for analysis of a detection target according to claim 6, wherein the nucleic acid aptamer specific to the detection target has a specific bindability to a specific molecule expressed on a surface of the microparticle having a membrane structure.

10. A method for analyzing a detection target, comprising:
a step of contacting a sample containing a detection target with the sensor for analysis of a detection target according to claim 6 to bind the detection target to the nucleic acid aptamer; and
a step of detecting a change in signal derived from the signal substance.

11. A manufacture method of a base material for producing a sensor for analysis of a detection target, comprising:
a monomolecular film formation step (1-1) of forming on a base material a monomolecular film having a polynucleotide group for nucleic acid aptamer's binding and a polymerization initiating group on a surface thereof;
a template introduction step (1-2) of introducing a template having on a surface thereof a polynucleotide group capable of complementary binding with the polynucleotide group for nucleic acid aptamer's binding, into the polynucleotide group for nucleic acid aptamer's binding;
a surface modification step (1-3) of modifying a surface of the template with a polymerizable functional group via a reversible linked group;
a polymerization step (1-4) of forming a polymer film on a surface of the base material by adding a polymerizable monomer and synthesizing a molecularly imprinted polymer corresponding to a part of the surface of the template using the polymerizable monomer and the polymerizable functional group as substrates and the polymerization initiating group as a polymerization initiator; and
a removal step (1-5) of cleaving the complementary binding and the reversible linked group to convert respectively into a polynucleotide group for nucleic acid aptamer's binding and a group for signal substance's binding, and removing the template.

12. The manufacture method of a base material for producing a sensor for analysis of a detection target according to claim 11, wherein the polynucleotide group for nucleic acid aptamer's binding has a length of 8 bases or more.

13. The manufacture method of a base material for producing a sensor for analysis of a detection target claim 11, wherein the polynucleotide group for nucleic acid aptamer's binding is a single chain.

14. The manufacture method of a base material for producing a sensor for analysis of a detection target according to claim 11, wherein the template has, on a surface thereof, a reversible binding group capable of forming the reversible linked group by binding to the group for signal substance's binding, together with the polynucleotide group for nucleic acid aptamer's binding.

15. The manufacture method of a base material for producing a sensor for analysis of a detection target according to claim 11, wherein the template is a silica particle.

16. The manufacture method of a base material for producing a sensor for analysis of a detection target according to claim 11, wherein the group for signal substance's binding is a thiol group, and a reversible bond group capable of forming the reversible linked group by binding to the group for signal substance's binding is a thiol group.

17. A manufacture method of a sensor for analysis of a detection target, comprising:
a step (1) of performing the manufacture method of a base material for producing a sensor for analysis of a detection target according to claim 11;
a step (2) of binding a nucleic acid aptamer specific to a detection target to the polynucleotide group for nucleic acid aptamer's binding by complementary binding; and
a step (3) of binding a signal substance to the group for signal substance's binding.


SEQUENCE LISTING (11 sequences)

SEQ ID NO: 1 - DNA, 32 bp, Artificial Sequence
  DNA aptamer specific for CD63
caccccacctcgctcccgtgacactaatgcta

SEQ ID NO: 2 - DNA, 25 bp, Artificial Sequence
  MUC-1 specific DNA aptamers
gcagttgatcctttggataccctgg

SEQ ID NO: 3 - DNA, 48 bp, Artificial Sequence
  EpCAM-specific DNA aptamers
cactacagaggttgcgtctgtcccacgttgtcatggggggttggcctg

SEQ ID NO: 4 - DNA, 54 bp, Artificial Sequence
  HER2-specific DNA aptamers
gggccgtcgaacacgagcatggtgcgtggacctaggatgacctgagtactgtcc

SEQ ID NO: 5 - DNA, 76 bp, Artificial Sequence
  DNA aptamer specific to ERa
ataccagcttattcaattcgttgcatttaggtgcattacgggggttatccgctctctcagatagtatgtgcaatca

SEQ ID NO: 6 - DNA, 51 bp, Artificial Sequence
  DNA aptamer specific for SARS-CoV-2
cagcaccgaccttgtgctttgggagtgctggtccaagggcgttaatggaca

SEQ ID NO: 7 - DNA, 67 bp, Artificial Sequence
  DNA aptamer specific for SARS-CoV-2
atccagagtgacgcagcatttcatcgggtccaaaaggggctgctcgggattgcggatatggacacgt

SEQ ID NO: 8 - DNA, 20 bp, Artificial Sequence
  Polynucleotide hybridizable to polynucleotide group for binding nucleic acid aptamer
cacaaatctgtcgctgagta

SEQ ID NO: 9 - DNA, 20 bp, Artificial Sequence
  polynucleotide group for binding nucleic acid aptamer
tactcagcgacagatttgtg

SEQ ID NO: 10 - DNA, 52 bp, Artificial Sequence
  polypeptide which gives nucleic acid aptamer
cacaaatctgtcgctgagtacaccccacctcgctcccgtgacactaatgcta

SEQ ID NO: 11 - DNA, 32 bp, Artificial Sequence
  Random polynucleotide without specificity for CD63
tgtgcggcgaaatattatagctaccgcaatta